(19) United States
(12) Patent Application Publication
(10) Pub. No.: US 2010/0172956 A1
(43) Pub. Date: Jul. 8, 2010

(54) DISSOLVING STRIP FOR ORAL MUCOSA AS A SYSTEMIC DRUG DELIVERY ROUTE

(76) Inventor: Thomas Kalili, Beverly Hills, CA (US)

Correspondence Address: BANNER & WITCOFF, LTD., TEN SOUTH WACKER DRIVE, SUITE 3000, CHICAGO, IL, 60606, US

(21) Appl. No.: 12/445,190

(22) PCT Filed: Oct. 12, 2007
(86) PCT No.: PCT/US07/81213

§ 371 Date: Feb. 19, 2010

Related U.S. Application Data
(60) Provisional application No. 60/829,460, filed on Oct. 13, 2006.

(30) Foreign Application Priority Data
Oct. 12, 2007 (US) PCT/US2007/081213

(51) Int. Cl. A61K 9/70; A61K 47/36

(52) U.S. Cl. 424/435; 514/777

(57) ABSTRACT

The invention relates to a dissolving strip for drug delivery providing a generally inert water soluble base material attached to the maxillary molars in order to deliver a therapeutic material upon activation by the parotid and other oral salivary glands. The composition of the dissolving strip is a generally inert base material, such as pullulan. The pullulan is impregnated with a therapeutic material to form a solid mixture to carry the therapeutic agent. The dissolving strip has sufficient flexibility to adhere to the 1-4 maxillary molars and acts as a reservoir to deliver therapeutic agents, such as an anti-smoking formula, a diet formula, a sexual enhancement formula, an oral freshness formula, prescription drugs, non-prescription drugs, herbs, or vitamins, or any combination of the previously listed. The dissolving strip is capable of delivering the therapeutic agents in a sustained or time released fashion.

This application claims the benefit of U.S. Provisional Application No. 60/829,460 filed Oct. 13, 2006, and PCT Application No. PCT/US2007/081213 filed Oct. 12, 2007, both of which are incorporated herein by reference in their entirety.

### BACKGROUND

Today, the generally preferred method of drug delivery, from the perspective of both patient and clinician, is via the oral route. Although transmucosal routes, i.e. the mucosal linings of the nasal, rectal, vaginal and ocular cavities provide alternate locations for drug delivery, poor patient acceptability associated with these sites limits these routes to local application use rather than systemic drug administration. In contrast, the oral cavity is highly acceptable to patients. In fact, the mucosa is robust, relatively permeable with a rich blood supply and it shows short recovery times after being stressed or damaged. Furthermore, the virtual lack of langerhans cells makes the oral mucosa tolerant to potential allergens and oral transmucosal drug delivery bypasses first pass effect and avoids pre-systemic elimination in the GI tract. For these reasons, the oral mucosal cavity is a very attractive site for systemic drug delivery.

While the prior art has addressed the problem of transferring medication to the patient through the oral cavity, problems associated with this type of drug delivery system continue to exist. For instance, while U.S. Pat. No. 5,166,233 attempts to alleviate the adverse feeling in the oral cavity, patients and users still experience some discomfort during the extended period in which the film is attached to the oral cavity.

Therefore, a need exists in the art for a therapeutic dissolving strip that dissolves quickly and efficiently and which alleviates the problems discussed above.

### SUMMARY

The summary is provided to introduce a selection of concepts in a simplified form that are further described below in the detailed description. This summary is not intended to identify

An aspect of the invention relates to a dissolving strip with a generally inert water soluble base material having a sufficient flexibility to adapt to areas of the oral cavity without causing permanent deformation.

In another aspect of the invention, a therapeutic material may be applied to the dissolving strip such that when the dissolving strip is applied to areas within the oral cavity such as the teeth, the therapeutic material may be released into the oral cavity.

In another aspect of the invention, the generally inert base material comprises an extra cellular bacterial polysaccharide, such as pullulan.

In a further aspect of the invention, a method of combining a therapeutic material with a generally inert base material that comprises the steps of providing an amount of pullulan and an amount of therapeutic agent is disclosed. The method may also comprise the step of mixing the pullulan with the therapeutic material and an amount of water to form a pullulan solution. The method may also comprise the steps of spreading the pullulan solution over a generally flat plate and drying the pullulan solution on that plate, and cutting the pullulan solution into at least two solid dissolving units. Furthermore, the method may also comprise the steps of storing the solid dissolving units in a dispensing container and accessing the dispensing container to remove at least one of the solid dissolving units. Moreover, the method may also comprise the steps of placing at least one of the solid dissolving units in a liquid medium and dissolving the pullulan to release flavoring material into the liquid medium.

In another aspect of the invention, a method of delivering a therapeutic agent to the buccal surface of 1-4 maxillary molars that comprises the step of applying the therapeutic material onto a dissolving strip, the dissolving strip having being generally flexible to adapt to 1 to 4 maxillary molars without permanent deformation. The method may also comprise the step of applying the dissolving strip to the 1 to 4 maxillary molars, such that the dissolving strip dissolves after allowing the therapeutic material to release the therapeutic agent into the oral cavity.

In another aspect of the invention, a method of delivering a therapeutic agent to the buccal surface of 1-4 maxillary molars that comprises the step of triggering salivary release from the parotid salivary gland.

### BRIEF DESCRIPTION OF THE DRAWINGS

FIG. 1 illustrates a perspective view of the dissolving strip in accordance with an aspect of the invention.

FIG. 2 illustrates a perspective view of the 1-4 maxillary molars in accordance with an aspect of the invention.

### DETAILED DESCRIPTION

The invention relates to a dissolving strip to deliver therapeutic agents, such as an anti-smoking formula, a diet formula, a sexual enhancement formula, an oral freshness formula, prescription drugs, non-prescription drugs, herbs, or vitamins, or any combination of the previously listed impregnated therein which, when adhered to 1-4 of the maxillary molars triggers the parotid and other oral salivary glands to release saliva and dissolve the dissolving strip thereby releasing the therapeutic agent into the oral cavity.

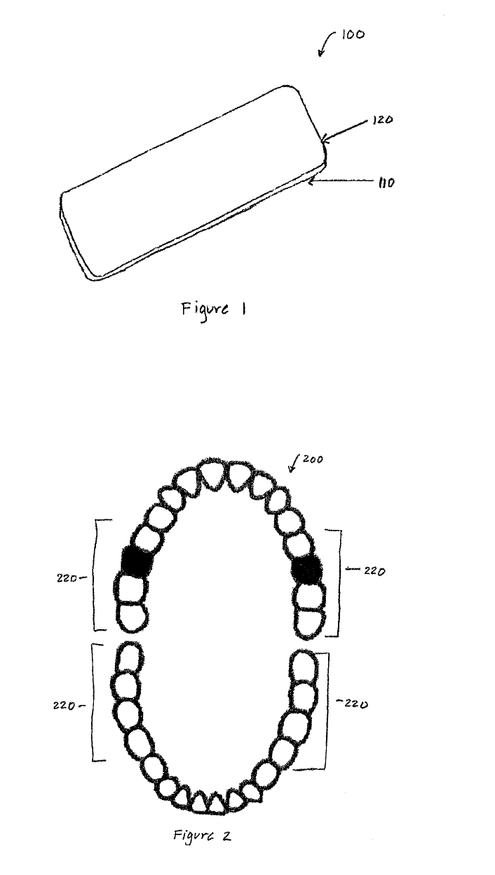

FIG. 1 illustrates a perspective view of the dissolving strip 100. In an aspect of the invention, a dissolving strip 100 and method of use thereof in which the dissolving strip 100 is constructed of an inert base material 110 such as pullulan or the like which, when impregnated with a therapeutic material 120 which will dissolve in the presence of salivary flow released from the Parotid and other oral salivary glands and trigger the release of an anti-smoking formula, a diet formula, a sexual enhancement formula, an oral freshness formula, prescription drugs, non-prescription drugs, herbs, or vitamins, or any combination of the previously listed. The release of said therapeutic material 120 will be into the oral cavity which is one of the most absorptive organs in our body for rapid therapeutic benefits of the therapeutic material 120.

In another aspect of the invention, the dissolving strip 100 may be placed in drinks where the inert base material 110 would be dissolved into the liquid medium to release the therapeutic material 120 and may be ingested to receive therapeutic benefits.

Moreover, another aspect of the invention provides a liquid flavoring to the dissolving strip 100 and method of use thereof which is relatively simple and economical in construction and is safe, efficient, and palatable in use.

FIG. 2 illustrates a perspective view of the 1-4 maxillary molars 200. In various aspects of the invention, a dissolving strip 100 may be applied to 1-4 maxillary molars 220 providing a generally inert base material 110 which is soluble in saliva and further providing a therapeutic material 120. A generally inert base material 110 is then impregnated with the therapeutic material 120 to form a solid mixture of the generally inert base material 110 and the therapeutic material 120 wherein the generally inert base material 110 functions to carry the therapeutic material 120 are then stored in a dispensing container. The generally inert base material 110 is then dissolved via the salivary medium to release the therapeutic material 120 into the oral cavity.

The substance may be in a viscous state, such as a gel, so that it provides the therapeutic material 120 while adhering to the 1-4 maxillary molars 220 due to its tackiness between the teeth surfaces and to hold the dissolving strip 100 in place. The conformable dissolving strip 100 may be of a size that individually fits over the 1-4 maxillary molars 220. As a soft, conformable material, the dissolving strip 100 may come into contact with the periodontium without causing physical irritation. The dissolving strip 100 may readily conform to the teeth by lightly pressing it on 1-4 maxillary molars 220 and/or by gently sucking through the gaps between teeth. The dissolving strip 100 may be readily conformable without permanent deformation to the shape of the teeth when the delivery system is placed on 1-4 maxillary molars 220. The dissolving strip 100 may readily dissolve within 30 seconds to 10 minutes of use.

By being a relatively thin coating, the therapeutic material 120 may be unobtrusive and manageable as compared to many who have difficulty with taking medications by; pill form, lozenger, suppositories, capsules and alternative remedies presently available for administrating herbal and or pharmaceutical medications into the body. Therefore, therapeutic substance may not be wasted with greater efficiency as to dosage, number of administrations, duration in order to meet proper recommended protocol. In an aspect of the invention, the dissolving strip 100 is substantially transparent so as to be almost unnoticeable when worn. Under conditions where the dissolving strip 100 is being utilized to administer therapeutic benefits for addictive conditions or circumstances where one may benefit from a reminder and discontinue their habitual patterns, the dissolving strip 100 may be ideal under such addictive conditions. The dissolving strip 100 may be a constant reminder for patients who are attempting to quit smoking or to lose weight representing a reminder for them to adhere to their regimen.

The delivery system may include a therapeutic material 120 applied to the dissolving strip 100. When the delivery system is placed on the surface of the teeth, salivary flow from the parotid gland and other oral glands may cause the dissolving strip 100 to dissolve releasing the therapeutic material 120 into the oral cavity. Due to the increased vasculature the rate of absorption may be 1,000 to 2,000 times greater than that of skin and one of the most rapid absorptive sites of the human body. Further, amongst the various routes of drug delivery, the oral route is preferred by patients and clinicians due to its ease of access and convenience of use as compared to transmucosal routes of drug delivery such as; the mucosal linings of the nasal, rectal, vaginal and/or ocular. Within the oral mucosal cavity, the buccal region adjacent to the maxillary molars offers an attractive route of administration for systemic therapeutic delivery. The mucosa has a rich blood supply and it is relatively permeable.

In an aspect of the invention, the delivery system may include a dissolving strip 100, which is initially substantially rectangular and flat, with rounded corners. Incorporated into the dissolving strip 100 may be a therapeutic material 120. The substance may be homogeneously, uniformly and continuously applied throughout the dissolving strip 100.

The delivery system may be applied to 1-4 maxillary molars 220 and the buccal surface of a tooth. Embedded in adjacent soft tissue are 1-4 maxillary molars 220. As those skilled in the art will realize, adjacent soft tissue may be defined as soft tissue surfaces surrounding the tooth structure including: papilla, marginal gingiva, gingival sulculus, inter dental gingiva, gingival gum structure on lingual and buccal surfaces up to and including mucoginival junction and the pallet and other surrounding surfaces.

The dissolving strip 100 may have a thickness and flexural stiffness which enable it to conform to the contoured surfaces of adjacent teeth and soft tissue. The dissolving strip 100 may have sufficient flexibility to adapt and adhere to 1 to 4 maxillary molars 220. The dissolving strip 100 may be readily conformable to tooth surfaces and to the interstitial tooth spaces without permanent deformation when the delivery system is applied. The delivery system may be applied without significant pressure.

In an aspect of the invention, the dissolving strip 100 is generally less than about 3 mm thick, preferably less than about 2 mm thick, and more preferably from about 0.01 to about 0.03 mm thick. A polyethylene dissolving strip 100 may be less than about 0.1 mm thick and more preferably from about 0.005 to about 0.02 mm thick. The shape of the dissolving strip 100 may be rectangular with rounded corners. “Rounded corners” may be defined as not having any sharp angles or points. Those skilled in the art will realize that the dissolving strip 100 may comprise other shapes and is not limited to being rectangular.

In an embodiment, the dissolving strip 100 may be of a size that individually fits over 1-4 maxillary molars 220 for maximum activation by the parotid salivary gland to release the therapeutic material 120. The therapeutic material 120 may be released into and absorbed by the oral mucosa, known to be one of the most absorptive anatomical sites of the human body. The length of the dissolving strip 100 may range from about 1 cm to about 8 cm and preferably from about 2 cm to about 6 cm. The width of the dissolving strip 100 may depend upon many factors, including whether or not the dissolving strip 100 wraps around the teeth and covers both surfaces of the tooth. In an embodiment, the width of the dissolving strip 100 is from about 0.5 cm to about 4 cm and preferably from about 1 to about 2 cm.

The relatively low stiffness may enable the dissolving strip 100 to adapt over the contoured surfaces of maxillary molars with very little force being exerted; that is, conformity to the curvature of the wearer's mouth and gaps between adjacent teeth is maintained because there is little residual force within the dissolving strip 100 to cause it to return to its substantially flat shape. The dissolving strip 100 may not require pressure foaming it against the teeth. The flexibility of the dissolving strip 100 may enable the dissolving strip 100 to contact adjoining soft tissue over an extended period of time up to several hours without physical irritation. Yet, the amount of time recommended for the dissolving strip 100 and thereby release the therapeutic material 120 into the oral cavity may range from 30 seconds to 10 minutes, preferably from 1 minute to 5 minutes before the dissolving strip 100 dissolves into the oral cavity.

The dissolving strip 100 is held in place on 1-4 maxillary molars 220 by the inherent characteristics of its substrate composition being pullulan. Pullulan is a natural polysaccharide and preferred to equivalent products of animal origin since the discovery of BSE (bovine spongiform encephalopathy). It is obtained through yeast fermentation of maize starch. It forms membranes easily, has adhesive properties and is highly soluble in water. The viscosity and general tackiness of the substance cause the dissolving strip 100 to be adhesively attached to 1-4 maxillary molars 220 without substantial slippage under the potential friction from the lips, tongue, and other soft tissue rubbing against the dissolving strip 100 during mouth movements associated with talking, drinking, etc.

Furthermore, the delivery system may be self-dissolving to release the therapeutic material 120 within a range of 30 seconds to 10 minutes and if necessary is easily removable from the surfaces of the teeth without the use of an instrument, chemical solvent, or undue friction. A peel force of from about 1 gram to about 10 grams for a 2 cm dissolving strip 100 width (approximately 5 grams/cm) may be all that is required.

In an aspect of the invention, the tooth surface may not be required to be prepared before the delivery system is applied. For example, the wearer may or may not choose to brush his/her teeth or rinse his/her mouth before applying the delivery system. The surfaces of the teeth may not be required to be dried or to be excessively wet with saliva or water before the dissolving strip 100 is applied.

The dissolving strip 100 may be substantially transparent so as to be almost unnoticeable when worn. Thinness of the delivery system 100 enables the higher temperature inside of the wearer's mouth to conduct heat through the dissolving strip 100 to the normally cooler teeth in order to accelerate the rate of diffusion of the therapeutic material 120 into the oral cavity.

The user may apply the delivery system to the teeth continuously for about 30 seconds to about 10 minutes a day, preferably from about 1 minute to about 5 minutes. Generally, this is daily as needed basis until the therapeutic activity desires are achieved. The amount of time and the number of days are dependent upon several factors, including the amount of therapeutic activity desired, the wearer's medical health and or psychological need.

The common method of manufacture of pullulan-based dissolving strips 100 is to dissolve the processed pullulan, which is an odorless white-colored powder, in an amount of water, then spread the film solution onto a Teflon-coated glass plate and dry it for a period of time. The resulting thin dried film is then cut into generally rectangular strips which will dissolve in liquids. Various other additives may be included with the pullulan strips, such as jelling agents, including carrageenan, alginate, or agar, in addition to other preservatives and materials to increase the shelf life of the pullulan-based dissolving strip 100. One or more of the therapeutic material 120 may be added to the dissolved pullulan material to form a pullulan solution prior to casting the solution onto the plate for drying and, when the resulting dried pullulan solution film is cut into strips, each of the strips would thus be flavored with flavoring. The packaging of the strips would then be completed in any appropriate form. The packaging, however, should be of a type which permits quick and simple dispensing of individual strips of the dissolving strips 100 of the dried pullulan solution to facilitate use.

In an aspect of the invention, a dissolving strip 100 for drug delivery providing a generally inert water soluble base material 110 attached to the 1-4 maxillary molars 220 in order to deliver a therapeutic material 120 upon activation by the parotid and other oral salivary glands may be provided. The composition of the dissolving strip 100 may be an inert base material 110 such as pullulan which is a natural polysaccharide and preferred to equivalent products of animal origin since the discovery of BSE (bovine spongiform encephalopathy). It forms membranes easily, exhibits adhesive properties and is highly soluble in water.

The pullulan may be impregnated with a therapeutic material 120 to form a solid mixture. Due to the oral cavity's increased vasculature it possesses superior absorptive advantages over other modes of drug ingestion. The dissolving strip 100 may have sufficient flexibility to adhere to maxillary posterior teeth and acts as a reservoir to deliver therapeutic agents, such as an anti-smoking formula, a diet formula, a sexual enhancement formula, an oral freshness formula, prescription drugs, non-prescription drugs, herbs, or vitamins, or any combination of the above.

The dissolving strip 100 may be readily conformable to the teeth surfaces without permanent deformation when applied to the teeth. The dissolving strip 100 may be attached to the 1-4 maxillary molars 220 (buccal/facial surface) adjacent to the parotid salivary gland which triggers release of the composition into the oral cavity. The dissolving strip 100 can be flavored with, for example, various types of mint, such as peppermint, spearmint, wintergreen and the like, or can be fruit or spice flavored, such as orange or cinnamon. The dissolving strip 100 may be capable of delivering drugs in sustained or time released fashion.

In an aspect of the invention, the delivery system may comprise a dissolving strip 100 having a sufficient flexibility to adapt and adhere to 1 to 4 maxillary molars 220 readily adaptable and conform to the tooth for comfort without permanent deformation when said delivery system is adhered to the maxillary molars. A therapeutic material 120 may be applied to the dissolving strip 100 such that when said delivery system is adhered to maxillary molars, the salivary gland, predominantly the parotid salivary gland, triggers release of the therapeutic material 120 into the oral cavity. The dissolving strip 100 may act as a reservoir and holds the therapeutic material 120 in place for a sufficient time to allow said therapeutic material 120 to be released into the oral cavity.

In another aspect of the invention, delivery of a therapeutic material 120 may be accomplished via the buccal surface of 1-4 maxillary molars 220. The method of delivery may include applying a substance onto a dissolving strip 100 having a sufficient flexibility to adapt and adhere to 1-4 maxillary molars 220 without permanent deformation; and applying the dissolving strip 100 with said substance thereon to said 1-4 maxillary molars 220 to dissolve with sufficient time to allow said therapeutic material 120 to be released into the oral cavity.

In another aspect of the invention, a delivery system for delivering a therapeutic material 120 which adapts and adheres to 1-4 maxillary molars 220 may be provided. The delivery system may include a dissolving strip 100 having sufficient flexibility to adapt and adhere on 1-4 maxillary molars 220, the dissolving strip 100 being readily conformable to tooth surfaces without permanent deformation when said delivery system is placed on 1-4 maxillary molars 220. The delivery system may be in an immediate proximity to the parotid salivary gland.

Upon adherence to 1-4 maxillary molars 220, a salivary release may be provided from the parotid salivary gland which will dissolve and activate the release of the therapeutic material 120 from the dissolving strip 100.

In another aspect of the invention, the delivery system may be adjacent to the buccal mucosa and secondarily the sublingual mucosa, two of the most vascularized anatomical structures in the human body. Due to the increased vasculature the rate of absorption is 1,000 to 2,000 times greater then that of skin and one of the most rapid absorptive sites of the human body thereby allowing efficient and rapid rate of absorption of the active for effective therapeutic results.

While particular embodiments of the present invention have been illustrated and described, it will be obvious to those skilled in the art that various changes and modifications may be made without departing from the spirit and scope of the invention.

I claim:

1. A dissolving strip comprising:
(a) an inert base material soluble in saliva; and
(b) a therapeutic material, wherein the inert base material is impregnated with the therapeutic material to form the dissolving strip, wherein the dissolving strip is dissolved into an oral cavity.

2. The dissolving strip of claim 1, wherein the time for the inert base to dissolve in saliva and thereby release the therapeutic material into the oral cavity ranges from 30 seconds to 10 minutes.

3. The dissolving strip of claim 1, wherein the inert base material comprises pullulan.

4. The dissolving strip of claim 1, wherein the therapeutic material comprises a therapeutic agent such as, an anti-smoking formula, a diet formula, a sexual enhancement formula, an oral freshness formula, a prescription drug, a non-prescription drug, a herb, a vitamin, or any combination thereof.

5. The dissolving strip of claim 1, wherein the therapeutic material is homogenously, uniformly and continuously applied throughout the dissolving strip.

6. The dissolving strip of claim 1, wherein the dissolving strip is placed in a liquid medium, wherein the inert base material is soluble in the liquid medium and wherein the therapeutic material is released into the liquid medium.

7. The dissolving strip of claim 1, wherein the dissolving strip is conformable to adhere to the surface of 1-4 maxillary molars.

8. The dissolving strip of claim 7, wherein the dissolving strip is in a gel to adhere to the surface of the 1-4 maxillary molars.

9. The dissolving strip of claim 7, wherein the dissolving strip includes a thickness and flexural stiffness to adapt and adhere to the surface of the 1-4 maxillary molars.

10. The dissolving strip of claim 7, wherein the dissolving strip further conforms to contoured surfaces of adjacent teeth and soft tissue.

11. The dissolving strip of claim 1, wherein the dissolving strip is substantially transparent.

12. The dissolving strip of claim 1, wherein the dissolving strip is substantially rectangular.

13. The dissolving strip of claim 1, wherein the dissolving strip is less than about 3 mm in thickness.

14. The dissolving strip of claim 1, wherein the length of the dissolving strip ranges from about 1 cm to about 8 cm.

15. The dissolving strip of claim 1, wherein the width of the dissolving strip ranges from about 0.5 cm to about 4 cm.

16. The dissolving strip of claim 1, wherein the dissolving strip further includes a liquid flavoring.

17. A method of delivering a therapeutic material through an oral cavity, the method comprising:
(a) applying the therapeutic material onto a dissolving strip having flexibility to adapt and adhere to 1-4 maxillary molars without permanent deformation; and
(b) applying the dissolving strip with the therapeutic material thereon to 1-4 maxillary molars to dissolve and release the therapeutic material into the oral cavity.

18. The method of claim 17, wherein the therapeutic material comprises a therapeutic agent such as an anti-smoking formula, a diet formula, a sexual enhancement formula, an oral freshness formula, a prescription drug, a non-prescription drug, a herb, a vitamin, or any combination thereof.

19. The method of claim 17, wherein the time the therapeutic material releases into the oral cavity ranges from 30 seconds to 10 minutes.

20. The method of manufacturing a dissolving strip, the method comprising:
(a) dissolving an inert base material in an amount of water to form a film solution;
(b) spreading the film solution onto a Teflon-coated glass plate;
(c) drying the film solution;
(d) cutting the dried film into generally rectangular strips to form inert base strips which will dissolve in liquid;
(e) adding additives to the inert base strips;
(f) adding a therapeutic material to the inert base strips to form a dissolving solution;
(g) casting the dissolving solution onto the Teflon-coated glass plate;
(h) drying the dissolving solution to form a dried dissolving solution; and
(i) cutting the dried dissolving solution into strips to form the dissolving strip.
